(19) United States
(12) Patent Application Publication
(10) Pub. No.: US 2003/0161818 A1
(43) Pub. Date: Aug. 28, 2003

(54) Cultures, products and methods using stem cells

(75) Inventors: Mark L. Weiss, Manhattan, KS (US); Deryl L. Troyer, Manhattan, KS (US); Duane Davis, Westmoreland, KS (US); Kathy E. Mitchell, Manhattan, KS (US)

Correspondence Address: MERCHANT & GOULD PC, P.O. BOX 2903, MINNEAPOLIS, MN, 55402-0903, US

(73) Assignee: Kansas State University Research Foundation

(21) Appl. No.: 10/083,779

(22) Filed: Feb. 25, 2002

(51) Int. Cl. A61K 48/00; C12N 5/08

(52) U.S. Cl. 424/93.21; 435/372; 514/44; 435/368

(57) ABSTRACT

Stem cells from human sources can have a variety of useful applications in disease treatment and biotechnology. More particularly the umbilical cord matrix stem (UCMS) cell cultures of the invention have a variety of totipotent, pluriotent, or multipotent cells for a variety of end uses from a non-controversial, universally available, species-specific source. The technology can have application to any placental animal, including agricultural and laboratory animals and humans. The invention relates to isolating, culturing the stem cells, maintaining the stem cells, transforming the stem cells into useful cell types using genetic or other transformation technologies, stem cell and tissue banking and using untransformed or transformed cells in disease treatment.

### FIELD OF THE INVENTION

The invention relates to the isolation and use of stem cells from mammalian species (potentially any placental animal including humans). More particularly the invention relates to obtaining stem cells that are at least multipotent and may be totipotent or nearly totipotent and are envisaged to have a variety of end uses. The cells are derived from a readily available source that is not controversial in humans or other animal applications. The invention also may be useful for providing a species-specific feeder cell layer or conditioned media for propagating embryonic stem cells. Invention relates to isolating the stem cells, culturing the stem cells, transforming the stem cells into useful cell types using genetic or other transformation technologies, and using untransformed or transformed cells in placental mammalian, human or animal disease treatment and related biotechnology.

### BACKGROUND OF THE INVENTION

### Stem Cells

Following fertilization of an egg by a sperm, a single cell is created that has the potential to form an entire differentiated multi-cellular organism including every differentiated cell type and tissue found in the body. This initial fertilized cell, with total potential is characterized as totipotent. Such totipotent cells have the capacity to differentiate into extra-embryonic membranes and tissues, embryonic tissues and organs. After several cycles (5 to 7 in most species) of cell division, these totipotent cells begin to specialize forming a hollow sphere of cells, the blastocyst. The inner cell mass of the blastocyst is composed of stem cells described as pluripotent because they can give rise to many types of cells that will constitute most of the tissues of an organism (not including some placental tissues etc.). Multipotent stem cells are more specialized giving rise to a succession of mature functional cells. The multipotent stem cell can give rise to hematopoietic, mesenchymal or neuroectodermal cell lines.

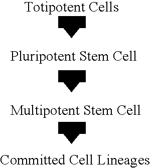

### Characteristics of Useful Pluripotent Stem Cells

True pluripotent stem cells should: (i) be capable of indefinite proliferation in vitro in an undifferentiated state; (ii) maintain a normal karyotype through prolonged culture; and (iii) maintain the potential to differentiate to derivatives of all three embryonic germ layers (endoderm, mesoderm, and ectoderm) even after prolonged culture. Strong evidence of these required properties have been published only for rodent embryonic stem cells (ES cells) and embryonic germ cells (EG cells) including mouse (Evans & Kaufman, Nature 292: 154-156, 1981; Martin, Proc Natl Acad Sci USA 78: 7634-7638, 1981) hamster (Doetschmanet al. Dev Biol 127: 224-227, 1988), and rat (Iannaccone et al. Dev Biol 163: 288-292, 1994), and less conclusively for rabbit ES cells (Gileset al. Mol Reprod Dev 36: 130-138, 1993; Graves & Moreadith, Mol Reprod Dev 36: 424-433, 1993). However, only established stem cell lines from the rat (Iannaccone, et al., 1994, supra) and the mouse (Bradley, et al., Nature 309: 255-256, 1984) have been reported to participate in normal development in chimeras.

### Stem Cells—Methods of Isolation

### a) Non-Human

U.S. Pat. No. 5,843,780 discloses a purified preparation of non-human primate embryonic stem cells comprising the steps of isolating a primate blastocyst, isolating cells from the inner cellular mass (ICM) of the blastocyst, plating the ICM cells on a fibroblast layer (wherein ICM-derived cell masses are formed) removing an ICM-derived cell mass and dissociating the mass into dissociated cells, replating the dissociated cells on embryonic feeder cells and selecting colonies with compact morphology containing cells with a high nucleus/cytoplasm ratio, and prominent nucleoli. The cells of the selected colonies are then cultured.

U.S. Pat. No. 6,107,543 is directed to a method for isolating cultured totipotent stem cells from domestic animals and to a process for the culture of isolated, totipotent stem cells from domestic animals that allows retrieval of large populations of stem cells and maintenance of both pluripotent cells and totipotent cells in culture. The embryonic stem cells are derived from the inner cell mass or earlier stages (i.e., morula) of the developing embryo which can be maintained in a way such that they can multiply but do not differentiate. When the cells are exposed to differentiating conditions, they are totipotent and can develop into all the tissues of the body. The “inner cell mass” is defined as a thicker accumulation of cells at one pole of the blastocyst. The cell culture system can be used for isolating and culturing totipotent stem cells of domestic animals. These cells can be used in genetic manipulation techniques.

U.S. Pat. No. 6,107,543 is directed to a method for transferring a nucleus from a cultured totipotent embryonic stem cell derived from an in vivo or in vitro produced embryo to a recipient oocyte and culturing the resulting nuclear transferred embryo in vitro or in vivo comprising collecting embryos from donor animals, isolating the inner cell mass from the embryos, dissociating the stem cells of the inner cell mass to form donor nuclear transfer stem cells, culturing the dissociated donor nuclear transfer stem cells, collecting and culturing recipient oocyte from donor animals or their products, enucleating the oocyte, transferring a single stem cell to the enucleated oocyte to form a nuclear transferred oocyte, and forming a viable single cell embryo from the nuclear transferred oocyte.

U.S. Pat. No. 5,639,618 provides a method of isolating a lineage specific stem cell in vitro, comprising: (a) transfecting a pluripotent embryonic stem cell with a construct comprising a regulatory region of a lineage specific gene operably linked to a DNA encoding a reporter protein; (b) culturing the pluripotent embryonic stem cell under conditions such that the pluripotent embryonic stem cell differentiates into a lineage specific stem cell; and (c) separating the cells which express the reporter protein from the other cells in the culture, the cell which expresses the reporter protein being an isolated lineage specific stem cell. A lineage specific stem cell can also be identified utilizing this method.

### (b) Human

Stem cells can be isolated from any known source of stem cells, including, but not limited to, bone marrow, both adult and fetal, mobilized peripheral blood (MPB) and umbilical cord blood. The use of umbilical cord blood is discussed, for instance, in Issaragrishi et al. (1995) N. Engl. J. Med. 332:367-369. Initially, bone marrow cells can be obtained from a source of bone marrow, including but not limited to, ilium (e.g. from the hip bone via the iliac crest), tibia, femora, spine, or other bone cavities. Other sources of stem cells include, but are not limited to, embryonic yolk sac, fetal liver, and fetal spleen. Other mature tissue sources have been proposed as sources of stem cells, however these tissues are as yet not demonstrated to be workable.

Human pluripotent cells have been developed from two sources with methods previously developed in work with animal models. Pluripotent stem cells have been isolated directly from the inner cell mass of human embryos (ES cells) at the blastocyst stage obtained from In Vitro Fertilization programs. Pluripotent stem cells (EG cells) have also been isolated from terminated pregnancies.

The proposal that stem cells be obtained from an embryo source (commonly fertilized egg cells from fertility clinics) remains ethically controversial. The controversy surrounding obtaining stem cells from newly fertilized human material has increased a need for obtaining useful stem cells from a non-controversial source. Accordingly a substantial need for obtaining stem cells having a powerful universal and versatile treatment capability is present.

Multipotent stem cells have been found in adult tissue. For example, blood stem cells, found in the bone marrow and blood stream of adults, continually replenish red blood cells, white blood cells and platelets. However as a source for therapeutically useful or pluripotent stem cells adults remain problematic. Stem cells have not been isolated from all body tissues. Even when present in a tissue, adult stem cells are often present in only minute numbers and are difficult to isolate and purify. There is evidence that such adult stem cells may not have the same capacity to adapt or proliferate or differentiate as younger cells obtained from blastocyst, fetal or neonatal sources. Research on the early stages of cell specialization may not be possible with more mature and specialized adult stem cells.

### Pluripotent Stem Cells—Applications

### i. Research

Pluripotent stem cells have a number of possible applications. Pluripotent stem cells could provide insight into the complex events of human development particularly the cellular decision-making process that results in cell specialization. This might suggest treatments for disorders of abnormal cell specialization such as cancer and birth defects. Generating pluripotent stem cells would be useful for generating transgenic non-human primates for models of specific human genetic diseases or for other purposes. Stem cells will allow the generation of models for any human genetic disease for which the responsible gene has been cloned. The human genome project will identify an increasing number of genes related to human disease, but will not always provide insights into gene function. Transgenic models will be essential for elucidating mechanisms of disease and for testing new therapies.

### ii. Drug Testing

Drug testing may benefit from a source of human pluripotent stem cells as new medications could be tested on human cell lines before animal and human research.

### iii. Cell Therapies

Many diseases are the result of disruption of cellular function or destruction of body tissues. Stem cells could be used in “cell therapies” to replace destroyed, non-functioning or abnormally functioning tissue. For example, recent studies have demonstrated that neural stem cells from the Central Nervous System (CNS) show tropism for specific diseased areas of the brain when grafted into animals. Neural stem cells from the CNS are rare, difficult to obtain and are not a feasible source of cells for applications in human medicine. In the mid-1990's, it was shown that embryonic stem cells from mice could be induced to form neurons and glia in vitro. If pluripotent stem cells can be stimulated to develop into specialized cells, they could be used to treat a range of Central Nervous System disorders such as Parkinson's and Alzheimer's disease, spinal cord injury, stroke, ALS, Hematopoietic Disorders such as sickle cell disease, leukemia, Cardiac Disorders, inborn metabolic and storage diseases and other diseases, for example, diabetes.

By manipulating culture conditions, stem cells can be induced to differentiate to specific cell types such as blood cells, neural cells or muscle cells to mention a few examples.

### iv. Tissue Growth and Transplantation

Transplantation of exogenous progenitor cells may provide a means to repopulate diseased tissues and organs. One source of exogenous progenitor cells has been Bone Marrow Stromal (BMS) cells. BMS cells are pluripotent cells that can differentiate into bone, cartilage, fat, muscle, tendon, neurons and many other tissues. BMS cells transplanted into rats with induced liver damage contribute to the formation of new hepatic oval cells that can further differentiate into hepatocytes and ductal epithelium. Bone marrow derived cells also ‘home’ in to damaged muscle in irradiated mice.

BMS cells injected intracerebroventricularly migrate extensively and differentiate into glial cells and neurons in neonatal mice. Spinal cord neural stem cells injected into the Central Nervous System (CNS) differentiate into neurons or glia depending upon the injection site. Like the ‘homing’ potential of BMS cells to damage e.g. liver or muscle, neural stem cells and embryonic neuroblasts have tropism for glioma or degenerating neurons in adult brains. Neuroblasts injected into cortical lesions differentiate into projection neurons containing the appropriate neurotransmitter and receptor phenotype.

While the technique of ‘Tissue transplantation’ has been utilized extensively in order to replace damaged organs or tissues, problems with the procedure continue to limit its use. Finding donors is a problem. Harvesting the tissue (or cells) involves an invasive procedure. The supply of tissue is limited and patients often have to wait for long intervals before an organ is available. Some organs cannot be transplanted. The recipient must be immuno-suppressed to a degree that can have undesirable side effects and furthermore makes the patient susceptible to infections. The use of fetal tissues has raised ethical concerns. Sophisticated banking or storing materials for transplant is necessary. Post-mitotic cells are not amenable to genetic manipulation.

In many applications, a strong need for culture technology capable of growing and maintaining stable or useful cultures of stem cells has been a highly desired end. Many current stem cell cultures are based on murine cell culture “feeder cell” technology. Non-species specific feeder cell technology reduces the value of stem cell cultures due to the foreign nature of the source of the feeder cell. This is true for number of reasons including the fact that such non-species specific feeder cells contain both foreign cells and foreign growth factors. Further, we believe that the use of non-species specific feeder cells in combination with different but desirable cultured cells cannot provide the optimum the growth conditions as species specific derived feeder cells. This issue is particularly relevant to agricultural animals, endangered species, laboratory animals and non-human primate cells. Still further, non-human feeder cell technology reduces the value of human derived stem cell cultures. This is true for number of reasons including the fact that such non-human feeder cells contain both non-human cells and non-human growth factors. Further, we believe that the use of non-human feeder cells in combination with human cultured cells cannot provide the optimum the growth conditions as human derived feeder cells.

A new feeder cell technology is needed to ensure that stem cells are not contaminated with cells, organelles, metabolic products, peptides, antibodies, etc. from another species and are grown or maintained with optimal growth conditions.

A method is necessary that would make stem cells, both pluripotent and multipotent, easy to procure particularly in a manner that provides powerful, universal and versatile treatment capability using a commonly available non-controversial stem cell source.

There have been attempts to solve these problems. Some organs may be harvested from cadavers. Bone marrow may be collected from the living, a procedure that is painful and invasive. There has to be donor-recipient tissue matching (allograft). Attempts have been made to use animal tissue. For example, Parkinson patients have received tissue grafts harvested from fetal pig brain. Such a xenograft is antigenic and the immune response may kill the graft.

### SUMMARY OF THE INVENTION

### Overview

Stem cells are capable of self-regeneration and can become lineage committed progenitors which are dedicated to differentiation and expansion into a specific lineage. As used herein, “stem cells” refers to progenitors to hematopoietic and non-hematopoietic cell types and virtually all cell types in the body.

The invention is directed to isolated and purified human or other placental animal's stem cells derived from Umbilical Cord Matrix Stem (UCMS) cells. Such matrix cells typically include extravascular cells, mucous-connective tissue (e.g., Wharton's Jelly) but typically do not include cord blood cells or related cells. The invention addresses the use of cells that can include stem cells and other potentially useful cells such as myofibroblasts. Any of these cells may provide a source for differentiated cells and can provide an important feeder environment for the establishment or maintenance of stem cell cultures. The invention also relates to a method for isolating, purifying and culturally expanding human or other placental animals umbilical matrix (UCMS) cells derived from umbilical cord tissue and to characterization of and uses for such cells. The present invention is also directed to various methods and devices for treating various medical conditions. The methods and devices of the invention utilize isolated umbilical matrix (UCMS) stem cells that under certain conditions, can be induced to differentiate into different cell lines. Human umbilical matrix (UCMS) stem cell compositions are provided which serve as the progenitors for all umbilical matrix (UCMS) stem cell lineages. The human stem cells of the invention can be used in the form of non-mitotic cells as a feeder cell collection.

### Stem Cells from Umbilical Cord

The present invention is directed to a method of obtaining stem cells from umbilical cord matrix sometimes called mesenchyme or Wharton's Jelly, a source of stem cells that is inexhaustible, inexpensive, substantially free of cord blood and does not use cord blood or related cells as a source for useful cells.

The method of stem cell isolation comprises the steps of providing non-blood tissue specimen from umbilical cord containing umbilical matrix (UCMS) stem cells, adding cells from the umbilical tissue specimen to a medium which contains factors that stimulate umbilical matrix (UCMS) stem cell growth without differentiation and allows, when cultured, for the selective adherence of the umbilical matrix (UCMS) stem cells to a substrate surface, culturing the specimen-medium mixture, and removing the non-adherent matter from the substrate surface.

Another aspect of the invention is the development of a bank of stem cells that can be tissue typed and banked and expanded as needed. Cells can be differentiated or genetically manipulated in vitro.

Another aspect of the invention is the development of cell populations that can be rendered mitotically inactive and then used as feeder cells for establishing and maintaining ES and EG cells from various species.

Yet another aspect of the invention is directed to a method for culture expanding the isolated and/or purified umbilical matrix (UCMS) umbilical cord derived stem cells. The method comprises the steps of providing a tissue specimen containing umbilical matrix (UCMS) stem cells, adding cells from the specimen to a medium that contains factors that stimulate umbilical matrix (UCMS) stem cell growth without differentiation and allows, when cultured, for the isolated umbilical matrix (UCMS) cells to expand.

A further aspect of the present invention relates to a kit for isolating umbilical matrix (UCMS) stem cells from an umbilical cord. The kit is comprised of a device to open the serosa of an umbilical cord. The kit is comprised of a medium containing a factor that can stimulate the growth of the umbilical matrix (UCMS) cells without differentiation.

A further aspect of the invention relates to cell culture technology using the stem cells of the invention in a non-mitotic form has a feeder cell in combination with other stem cells capable of growth, transformation and use in treating human disease.

A further aspect of the invention relates to cell culture technology using the stem cells of the invention in a treatment for diseases such as myelomonoblastic leukemia.

A further aspect of the invention relates to cell culture technology using the stem cells of the invention in a treatment using the homing potential of the UCMS cell.

Utilization of Umbilical Cord (UCMS) Stem Cells Umbilical Cord Stem Cell (UCMS) produced by the present invention have a range of possible uses (in all placental animals, such uses including a homing potential in which the cells proceed to the site including but not limited to:

1) Regenerating UCMS tissues which have been damaged through acquired or genetic disease;

2) Treating a patient with damaged tissue or organs with umbilical derived UCMS Cells combined with a biocompatible carrier suitable for delivering UCMS Cells to the damaged tissue sites for correcting, repairing or modifying connective tissue disorders such as the regeneration of damaged skeletal muscle;

3) Producing various UCMS derived tissues;

4) Detecting, evaluating and isolating growth factors relevant to umbilical derived UCMS Cells self-regeneration and differentiation into specific UCMS lineages;

5) Detecting, evaluating and isolating inhibitory factors which modulate umbilical derived UCMS Cells commitment and differentiation into specific UCMS lineages;

6) Applying an umbilical derived UCMS cell to an area of connective tissue damage under conditions suitable for differentiating the cells into the type of connective tissue necessary for repair;

7) Developing UCMS cell lineages and assaying for factors associated with UCMS differentiation into various tissue types;

8) Various methods or devices for utilizing the umbilical derived UCMS cells in order to enhance hematopoietic cell production; and

9) Methods for using composite grafts of umbilical derived UCMS cells during bone marrow transplantation.

10) Methods for establishing and maintaining placental animal, including human, stem cell cultures using the Wharton's jelly derived stem cells as a species specific “feeder cell.”

For the purpose of this disclosure, the term “feeder cell” or “feeder cell culture”, as used herein, refers to cells that provide a co-stimulating function in conjunction with typically the other stem cell cultures, not necessarily the cells of this invention. A feeder cell can be obtained by culture techniques known in the art such as that shown by Weaver et al., Blood 82:1981-1984, 1993. Feeder cell cultures can be stored by cryopreservation in liquid nitrogen until use. Prior to the use of such feeder cells, for the purpose of maintaining a culture of stem cells (other than the feeder cells), such feeder cells are stabilized to promote the isolation and maintenance of stem cell cultures. “Homing potential” refers to an inherent capacity of a cell to be targeted to specific locations for therapeutic function or purpose.

### DETAILED DESCRIPTION OF THE INVENTION

### Summary

The present invention relates to a method for obtaining stem cells from umbilical cord matrix (e.g., Wharton's Jelly) a umbilical cord mucous connective tissue, involving:

1) Methods for isolating UCMS cells from umbilical cord matrix (e.g.) Wharton's Jelly of the umbilical cord;

2) Methods for mitotically expanding the populations of isolated UCMS cells, collectively the cells of the invention; and

3) Methods for culturing mitotically expanded populations of the cells of the invention under conditions that permit or induce the formation of new tissue.

The invention also relates to the products of these methods, including but not limited to, the cells of the invention, mitotically expanded or otherwise and the new tissue produced therefrom. The invention also relates to the use of these cells, constructs and tissues in vivo to repair, replace or augment tissues or organs of the animal or human or, in vitro, to form tissue cultures which are useful to produce new tissue or bioactive agents or to test the therapeutic or cytotoxic effects of potential therapeutic agents.

In addition, the cells of the invention can be cryopreserved and stored frozen. By this process, “banks” of cells that can be used to produce new tissue at any time to replace that lost to disease or trauma.

For supplying cell or tissue grafts, the cells of the invention could be used in two ways. Either the cells of an individual could be obtained and cryopreserved to be used at any time in the subject's life to replace damaged or diseased tissue or placed in a bank for use as “ubiquitous donor cells” or “cells with a homing potential” to produce tissue for use in any subject in need.

The cells of this invention could be used as feeders, feeder cells or feeder cultures to support stem cells or sources of conditioned media or extra cellular matrix to support stem cells of various species. The feeders might be of the same or a different species as the targeted stem cells.

### Definitions

### Umbilical Derived UCMS Cells

The term “Umbilical Cord Matrix Cell” as used herein refers to either:

1) A pluripotent, or lineage-uncommitted progenitor cell, typically referred to in the art as a “stem cell” derived from the umbilical cord matrix, other than a cord blood cell source. Such a cell is potentially capable of an unlimited number of mitotic divisions to either renew its line or to produce progeny cells which will differentiate into the mature functional cells that will constitute most of the tissues of an organism such as hematopoietic, mesenchymal or neuroectodermal cell lines; or

2) A lineage-committed progeny cell produced from the mitotic division of a stem cell of the invention that can eventually differentiate into hematopoietic, mesenchymal or neuroectodermal cells. Unlike the stem cell from which it is derived, the lineage-committed progeny cell is generally considered to be incapable of an unlimited number of mitotic divisions to produce other progeny cells.

The invention is directed primarily to compositions and methods for the production of umbilical derived UCMS cells and their derivatives such as hematopoietic, mesenchymal or neuroectodermal cell lines and cells, tissues and organs in humans. However the invention may also be practiced so as to produce stem cells and their derivatives in any mammal in need thereof.

According to the invention, stem cells may be obtained from UCMS cell source such as Wharton's jelly collected from a subject's own umbilical cord. Alternatively, it may be advantageous to obtain stem cells from Wharton's jelly obtained from an umbilical cord associated with a species specific or species related developing fetus or newborn, where the subject in need of treatment is one of the parents of the fetus or newborn. Another scenario involves banking and tissue typing and cataloging so that any individual in need of a stem cell graft might find an appropriate match.

Alternatively, because of the primitive nature of cells isolated from Wharton's jelly, immune rejection of the cells of the invention or the new tissue produced therefrom may be minimized. As a result, such cells may be useful as “ubiquitous donor cells” for the production of new cells and tissue for use in any subject in need thereof.

### “Wharton's Jelly”

The term “Wharton's Jelly,” also known as inter-laminar jelly, as used herein, refers to a mucous-connective tissue substance found in the umbilical cord. The components of Wharton's Jelly include a mucous connective tissue in which are found myfofibroblasts, fibroblasts, collagen fibers and an amorphous ground substance composed of hyaluronic acid and possibly other as yet uncharacterized cell populations. Wharton's jelly is one component of the umbilical cord matrix and can be a source of the stem cells used in the invention.

### DESCRIPTION OF THE INVENTION

The invention is divided into the following non-limiting sections solely for the purpose of description:

1) Obtaining umbilical cord;

2) Method of obtaining UCMS cells from Wharton's Jelly;

3) Establishing and maintaining stem cells to a cell culture;

4) Establishing the stem cells into a transplantable cell, including cells with a homing capacity;

5) Foreign gene introduction;

6) Development of a stem cell bank; and

7) Development of species specific or other appropriate feeder culture cells for ES, EG or other stem cells (for example, neural stem cells).

### (1) Obtaining Umbilical Cord

In order to isolate the stem cells according to the invention, umbilical cord is obtained under sterile conditions immediately following the termination of pregnancy (either full term or pre-term). The umbilical cord or a section thereof, according to one embodiment of the invention, may be transported from the site of the delivery to a laboratory in a sterile container containing a preservative medium. One example of such a preservative medium is Dulbecco's Modified Eagle's Medium (DMEM) with hepes buffer.

The umbilical cord is preferably maintained and handled under sterile conditions prior to and during the collection of the stem cells from the matrix or Wharton's jelly and may additionally be surface-sterilized by brief surface treatment of the cord with, for example, an aqueous (70% ethanol) solution or betadine, followed by a rinse with sterile, distilled water. The umbilical cord can be briefly stored for up to about three hours at about 3-5° C., but not frozen, prior to extraction of UCMS cell(s) from the cellular source including the Wharton's Jelly umbilical component.

Wharton's jelly is collected from the umbilical cord under sterile conditions by an appropriate method known in the art. For example, the cord is cut transversely with a scalpel, for example, into approximately one inch sections, and each section is transferred to a sterile container containing a sufficient volume of phosphate buffered saline (PBS) containing CaCl₂ (0.1 g/l) and MgCl₂.6H₂O (0.1 g/l) to allow surface blood to be removed from the section by gentle agitation. The section is then removed to a sterile-surface where the outer layer of the section is sliced open along the cord's longitudinal axis. The blood vessels of the umbilical cord (two veins and an artery) are dissected away, for example, with sterile forceps and dissecting scissors, and the Wharton's jelly is collected and placed in a sterile container, such as a 100 mm TC-treated Petri dish. The Wharton's jelly may then be cut into smaller sections, such as 2-3 mm³ for culturing.

### (2) Method of Obtaining UCMS Cells from Wharton's Jelly

Wharton's jelly is incubated in vitro in culture medium under appropriate conditions to permit the proliferation of any UCMS cells present therein. Any appropriate type of culture medium can be used to isolate the stem cells of the invention, such as, but not limited to DMEM. The culture medium may be supplemented with one or more components including, for example, fetal bovine serum, equine serum, HUMAN SERUM and one or more antibiotics and/or mycotics to control microbial contamination. Examples of antibiotics include but are not limited to penicillin G, streptomycin sulfate, amphotericin B, gentamycin, and nystatin, either alone or in combination.

Methods for the selection of the most appropriate culture medium, medium preparation, and cell culture techniques are well known in the art and are described in a variety of sources, including Doyle et al., (eds.), 1995, Cell and Tissue Culture: Laboratory Procedures, John Wiley & Sons, Chichester; and Ho and Wang (eds.), 1991, Animal Cell Bioreactors, Butterworth-Heinemann, Boston, which are incorporated herein by reference.

Another method relies on enzymatic dispersion of Wharton's Jelly with collagenase and isolation of cells by centrifugation followed by plating.

### (3) Establishment of UCMS Cells in Cell Culture

The method involves fractionating the source of cells (Wharton's Jelly) into two fractions, one of which is enriched with a stem cell and thereafter exposing the stem cells to conditions suitable for cell proliferation. The cell enriched isolate thus created comprises totipotent immortal stem cells.

After culturing Wharton's jelly for a sufficient period of time, for example, about 10-12 days, UCMS derived stem cells present in the explanted tissue will tend to have grown out from the tissue, either as a result of migration therefrom or cell division or both. These UCMS derived stem cells may then be removed to a separate culture vessel containing fresh medium of the same or a different type as that used initially, where the population of UCMS derived stem cells can be mitotically expanded.

Alternatively, the different cell types present in Wharton's jelly can be fractionated into subpopulations from which UCMS derived stem cells can be isolated. This may be accomplished using standard techniques for cell separation including, but not limited to, enzymatic treatment to dissociate Wharton's jelly into its component cells, followed by cloning and selection of specific cell types (for example, myofibroblasts, stem cells, etc.), using either morphological or biochemical markers, selective destruction of unwanted cells (negative selection), separation based upon differential cell agglutinability in the mixed population as, for example, with soybean agglutinin, freeze-thaw procedures, differential adherence properties of the cells in the mixed population, filtration, conventional and zonal centrifugation, centrifugal elutriation (counter-streaming centrifugation), unit gravity separation, countercurrent distribution, electrophoresis, and fluorescence activated cell sorting (FACS). For a review of clonal selection and cell separation techniques, see Freshney, 1994, Culture of Animal Cells; A Manual of Basic Techniques, 3d Ed., Wiley-Liss, Inc., New York, which is incorporated herein by reference.

In a preferred embodiment for culturing UCMS derived stem cells, Wharton's jelly is cut into sections of approximately 2-3 mm³, and placed in a TC-treated Petri dish containing glass slides on the bottom of the Petri dish. The tissue sections are then covered with another glass slide and cultured in a complete medium, such as, for example, RPMI 1640 containing 10% FBS, 5% ES and antimicrobial compounds, including penicillin G (100 μg/ml), streptomycin sulfate (100 μg/ml), amphotericin (250.mu.g/ml), and gentamicin (10 μg/ml), pH 7.4-7.6. The tissue is preferably incubated at 37-39 degrees. C. and 5% CO₂ for 10-12 days.

The medium is changed as necessary by carefully aspirating the medium from the dish, for example, with a pipette, and replenishing with fresh medium. Incubation is continued as above until a sufficient number or density of cells accumulates in the dish and on the surfaces of the slides. For example, the culture obtains approximately 70 percent confluence but not to the point of complete confluence. The original explanted tissue sections may be removed and the remaining cells are trypsinized using standard techniques. After trypsinization, the cells are collected, removed to fresh medium and incubated as above. The medium is changed at least once at 24 hr post-trypsin to remove any floating cells. The cells remaining in culture are considered to be UCMS derived stem cells.

Once the stem cells have been isolated, their population is expanded mitotically. The stem cells should be transferred or “passaged” to fresh medium when they reach an appropriate density, such as 3×10⁴-cm⁻² to 6.5×10⁴-cm⁻², or, for example, when they reach a defined percentage of confluency on the surface of a culture dish. During incubation of the stem cells, cells can stick to the walls of the culture vessel where they can continue to proliferate and form a confluent monolayer. Alternatively, the liquid culture can be agitated, for example, on an orbital shaker, to prevent the cells from sticking to the vessel walls. The cells can also be grown on Teflon-coated culture bags.

In a preferred embodiment, the desired mature cells or cell lines are produced using stem cells that have gone through a low number of passages. We, however, have maintained cells for more than 55 passages and at least 60 doublings. The invention contemplates that once stem cells have been established in culture, their ability to serve as progenitors for mature cells or cell lines can be maintained, for example, by regular passage to fresh medium as the cell culture reaches an appropriate density or percentage of confluency, or by treatment with an appropriate growth factors, or by modification of the culture medium or culture protocol, or by some combination of the above.

### (4) Establishing the Stem Cell into a Transplantable Culture

The invention also includes a method of developing transplantable cells by exposing the stem cells to differentiating or growth factors. The transplantable cell may be a hematopoietic cell, a mesenchymal cell or a neuroectodermal cell, a neural cell or other cell.

Once established, a culture of UCMS derived stem cells may be used to produce mature cells or cell lines. Differentiation of stem cells to mature cells can be triggered by the addition to the culture medium of specific exogenous growth factors, such as, for example, bFgF BMPs such as BMP-13 or TGF-β, with or without antioxidants.

### (5) Foreign Gene Introduction

The invention also includes a method of introducing a foreign gene into a stem cell by contacting the stem cell with a factor comprising a foreign gene. Stem cells can be genetically engineered to express genes for specific types of growth factors.

In a non-limiting embodiment, the cells of the invention, for example, may be genetically engineered to express and produce growth factors such as BMP-13 or TGF-β. For example, the gene or coding sequence for TGF-β would be placed in operative association with a regulated promoter so that production of TGF-β in culture can be controlled. If desired, the cells of the invention may be genetically engineered to produce other gene products beneficial to transplantation, e.g., anti-inflammatory factors, e.g., anti-GM-CSF, anti-TNF, anti-IL-1, anti-IL-2, etc.

Alternatively, the cells may be genetically engineered to “knock out” expression of native gene products that promote inflammation, e.g., GM-CSF, TNF-α, IL-1, IL-2, or “knock out” expression of MHC in order to lower the risk of rejection. In addition, the cells may be genetically engineered for use in gene therapy to adjust the level of gene activity in a patient to assist or improve the results of tissue transplantation. The genetically engineered cells may then be screened to select those cell lines that: 1) bring about the amelioration of symptoms of rheumatoid disease or inflammatory reactions in vivo, and/or 2) escape immunological surveillance and rejection.

### (6) Stem Cell Bank

The invention includes a method of generating a bank of mammalian totipotent stem cells by obtaining mesenchyme cells from the umbilical cord, fractionating the mesenchyme into a fraction enriched with a stem cell and culturing the stem cells in a culture medium containing one or more growth factors. By this process, the stem cells will undergo mitotic expansion.

The invention contemplates the establishment and maintenance of cultures of stem cells as well as mixed cultures comprising stem cells, mature cells and mature cell lines. Once a culture of stem cells or a mixed culture of stem cells and mature cells is established, the cultures should be transferred to fresh medium when sufficient cell density is reached. By this means, formation of a monolayer of cells should be prevented or minimized, for example, by transferring a portion of the cells to a new culture vessel and into fresh medium. Alternatively, the culture system can be agitated prevent the cells from sticking or grown in Teflon-coated culture bags.

Once the cells of the invention have been established in culture, as described above, they may be maintained or stored in “cell banks” comprising either continuous in vitro cultures of cells requiring regular transfer, or, preferably, cells which have been cryopreserved.

Cryopreservation of cells of the invention may be carried out according to known methods, such as those described in Doyle et al., 1995, Cell and Tissue Culture. For example, but not by way of limitation, cells may be suspended in a “freeze medium” such as, for example, culture medium further comprising 15-20% FBS and 10% dimethylsulfoxide (DMSO), with or without 5-10% glycerol, at a density, for example, of about 4-10×10⁶ cells-ml⁻¹. The cells are dispensed into glass or plastic ampoules (Nunc) that are then sealed and transferred to the freezing chamber of a programmable freezer. The optimal rate of freezing may be determined empirically. For example, a freezing program that gives a change in temperature of about −1 degree. C.-min⁻¹ through the heat of fusion may be used. Once the ampoules have reached about −180 degree. C., they are transferred to a liquid nitrogen storage area. Cryopreserved cells can be stored for a period of years, though they should be checked at least every 5 years for maintenance of viability.

The cryopreserved cells of the invention constitute a bank of cells, portions of which can be “withdrawn” by thawing and then used to produce new stem cells, etc. as needed. Thawing should generally be carried out rapidly, for example, by transferring an ampoule from liquid nitrogen to a 37 degree C. water bath. The thawed contents of the ampoule should be immediately transferred under sterile conditions to a culture vessel containing an appropriate medium such as RPMI 1640, DMEM conditioned with 20% FBS. The cells in the culture medium are preferably adjusted to an initial density of about 3×10⁵ cells-ml⁻¹-6×10⁵ cells-ml⁻¹ so that the cells can condition the medium as soon as possible, thereby preventing a protracted lag phase. Once in culture, the cells may be examined daily, for example, with an inverted microscope to detect cell proliferation, and sub-cultured as soon as they reach an appropriate density.

The cells of the invention may be withdrawn from the bank as needed, and used for the production of new tissue either in vitro, or in vivo, for example, by direct administration of cells to the site where new tissue is needed. As described supra, the cells of the invention may be used to produce new tissue for use in a subject where the cells were originally isolated from that subject's umbilical cord (autologous).

Alternatively, the cells of the invention may be used as ubiquitous donor cells, i.e., to produce new tissue for use in any subject (heterologous).

### Uses of the UCMS Derived Stem Cells

The cells of the invention may be used to treat subjects requiring the repair or replacement of body tissues resulting from disease or trauma. Treatment may entail the use of the cells of the invention to produce new tissue, and the use of the tissue thus produced, according to any method presently known in the art or to be developed in the future. For example, the cells of the invention may be implanted, injected or otherwise administered directly to the site of tissue damage so that they will produce new tissue in vivo.

In addition, the umbilical cord mesenchyme derived stem cells, the mature cells produced from these stem cells, the cell lines derived from these stem cells and the tissue of the invention can be used:

(1) to screen for the efficacy and/or cytotoxicity of compounds, allergens, growth/regulatory factors, pharmaceutical compounds, etc.;

(2) to elucidate the mechanism of certain diseases;

(3) to study the mechanism by which drugs and/or growth factors operate;

(4) to diagnose, monitor and treat cancer in a patient;

(5) for gene therapy; and

(6) to produce biologically active products, to name but a few uses

(7) to target delivery of a drug to a specific tissue. To do this they may first be engineered to produce the drug; and

(8) to be utilized for their homing ability that permits the cells to migrate from a treatment location to a specific target location (for example, where a pathology or abnormal condition exists).

### (1) Screening Effectiveness and Cytotoxicity of Compounds

The cells and tissues of the invention may be used in vitro to screen a wide variety of compounds for effectiveness and cytotoxicity of pharmaceutical agents, growth/regulatory factors, anti-inflammatory agents, etc. To this end, the cells of the invention, or tissue cultures described above, are maintained in vitro and exposed to the compound to be tested. The activity of a cytotoxic compound can be measured by its ability to damage or kill cells in culture. This may readily be assessed by vital staining techniques. Analyzing the number of living cells in vitro, e.g., by total cell counts, may assess the effect of growth/regulatory factors and differential cell counts. This may be accomplished using standard cytological and/or histological techniques, including the use of immunocytochemical techniques employing antibodies that define type-specific cellular antigens. The effect of various drugs on the cells of the invention either in suspension culture or in the three-dimensional system described above may be assessed.

### (2) Elucidate the Mechanism of Certain Diseases

The cells and tissues of the invention may be used as model systems for the study of physiological or pathological conditions. For example, the cells and tissues of the invention may be used to determine the nutritional requirements of a tissue under different physical conditions, e.g., intermittent pressurization, and by pumping action of nutrient medium into and out of the tissue construct. This may be especially useful in studying underlying causes for age-related or injury-related disorders.

### (3) Study the Mechanism by Which Drugs and/or Growth Factors Operate

The stem cells, cell lines, mature cells and tissues of the invention may also be used to study the mechanism of action of cytokines and other pro-inflammatory mediators, e.g., IL-1, TNF and prostaglandins. In addition, cytotoxic and/or pharmaceutical agents can be screened for those that are most efficacious for a particular patient. Agents which prove to be efficacious in vitro could then be used to treat the patient therapeutically.

### (4) Diagnosis, Monitoring and Treatment of Cancer or Cancer Cells, Tissues or Symptoms

Based upon their tropism for tissue damage, the cells and tissues of the invention may be used to diagnose, treat or monitor cancer or reduce its symptoms.

### (5) Gene Therapy

The cells and tissues of the present invention may afford a vehicle for introducing genes and gene products in vivo to assist or improve the results of implantation and/or for use in gene therapies. The following description is directed to the genetic engineering of any of the cells of the invention or tissues produced therefrom.

Cells which express a gene product of interest, or the tissue produced in vitro therefrom, can be implanted into a subject who is otherwise deficient in that gene product. For example, genes that express a product capable of preventing or ameliorating symptoms of various types of diseases, such as those involved in preventing inflammatory reactions, may be under-expressed or down-regulated under disease conditions. Alternatively, the activity of gene products may be diminished, leading to the manifestation of some or all of the pathological conditions associated with a disease. In either case, the level of active gene product can be increased by gene therapy, i.e., by genetically engineering cells of the invention to produce active gene product and implanting the engineered cells, or tissues made therefrom, into a subject in need thereof. A related application foreseen in agricultural or other animals is the delivery of a product that enhances growth, maturation, reproduction, etc. The products of interest may be delivered over the long term or alternatively and transiently to achieve the desired effect.

Alternatively, the cells of the invention can be genetically engineered to produce a gene product that would serve to stimulate tissue or organ production such as, for example, BMP-13 or TGF-β. Also, for example, the cells of the invention may be engineered to express the gene encoding the human complement regulatory protein that prevents rejection of a graft by the host. See, for example, McCurry et al., 1995, Nature Medicine 1:423-427.

A related application foreseen in animals is the use of these cells to generate transgenic animals using methods that have been developed for mouse ES cells. The chimeric animals will be used to establish transgenic animal lines. Another related application foreseen in animals is the use of these cells to generate chimeric animals that produce useful compounds.

Methods that may be useful to genetically engineer the cells of the invention are well-known in the art. For example, a recombinant DNA construct or vector containing the gene of interest may be constructed and used to transform or transfect one or more cells of the invention. Such transformed or transfected cells that carry the gene of interest, and that are capable of expressing said gene, are selected and clonally expanded in culture. Methods for preparing DNA constructs containing the gene of interest, for transforming or transfecting cells, and for selecting cells carrying and expressing the gene of interest are well-known in the art. See, for example, the techniques described in Maniatis et al., 1989, Molecular Cloning, A Laboratory Manual, Cold Spring Harbor Laboratory Press, Cold Spring Harbor, N.Y.; Ausubel et al., 1989, Current Protocols in Molecular Biology, Greene Publishing Associates & Wiley Interscience, N.Y.; and Sambrook et al., 1989, Molecular Cloning: A Laboratory Manual, 2nd Ed., Cold Spring Harbor Laboratory Press, Cold Spring Harbor, N.Y. In addition, the transkaryotic implantation technique described by Seldon et al., 1987, Science 236:714-718, may be useful. All of these publications are incorporated herein by reference.

The cells of the invention can be engineered using any of a variety of vectors including, but not limited to, integrating viral vectors, e.g., retrovirus vector or adeno-associated viral vectors, or non-integrating replicating vectors, e.g., papilloma virus vectors, SV40 vectors, adenoviral vectors; or replication-defective viral vectors. Other methods of introducing DNA into cells include the use of liposomes, electroporation, a particle gun, or by direct DNA injection.

Host cells are preferably transformed or transfected with DNA controlled by, i.e., in operative association with, one or more appropriate expression control elements such as promoter or enhancer sequences, transcription terminators, polyadenylation sites, among others, and a selectable marker. Following the introduction of the foreign DNA, engineered cells may be allowed to grow in enriched media and then switched to selective media. The selectable marker in the foreign DNA confers resistance to the selection and allows cells to stably integrate the foreign DNA as, for example, on a plasmid, into their chromosomes and grow to form foci which, in turn, can be cloned and expanded into cell lines. This method can be advantageously used to engineer cell lines that express the gene product.

Any promoter may be used to drive the expression of the inserted gene. For example, viral promoters include but are not limited to the CMV promoter/enhancer, SV 40, papillomavirus, Epstein-Barr virus, elastin gene promoter and β-globin. Preferably, the control elements used to control expression of the gene of interest should allow for the regulated expression of the gene so that the product is synthesized only when needed in vivo. If transient expression is desired, constitutive promoters are preferably used in a non-integrating and/or replication-defective vector. Alternatively, inducible promoters could be used to drive the expression of the inserted gene when necessary. Inducible promoters include, but are not limited to, those associated with metallothionein and heat shock protein.

Examples of transcriptional control regions that exhibit tissue specificity which have been described and could be used include but are not limited to: elastase I gene control region, which is active in pancreatic acinar cells (Swit et al., 1984, Cell 38:639-646; Ornitz et al., 1986, Cold Spring Harbor Symp. Quant. Biol. 50:399-409; MacDonald, 1987, Hepatology 7:425-515); insulin gene control region, which is active in pancreatic beta cells (Hanahan, 1985, Nature 315:115-122); immunoglobulin gene control region, which is active in lymphoid cells (Grosschedl et al., 1984, Cell 3S:647-658; Adams et al., 1985, Nature 318:533-538; Alexander et al., 1987, Mol. Cell. Biol. 7:1436-1444); myelin basic protein gene control region, which is active in oligodendrocyte cells in the brain (Readhead et al., 1987, Cell 48:703-712); myosin light chain-2 gene control region, which is active in skeletal muscle (Shani, 1985, Nature 314:283-286); and gonadotropic releasing hormone gene control region, which is active in the hypothalamus (Mason et al., 1986, Science 234:1372-1378).

The cells of the invention may be genetically engineered to “knock out” expression of factors that promote inflammation or rejection at the implant site. Negative modulatory techniques for the reduction of target gene expression levels or target gene product activity levels are discussed below. “Negative modulation,” as used herein, refers to a reduction in the level and/or activity of target gene product relative to the level and/or activity of the target gene product in the absence of the modulatory treatment. The expression of a gene native to a specific cell can be reduced or knocked out using a number of techniques including, for example, inhibition of expression by inactivating the gene completely (commonly termed “knockout”) using the homologous recombination technique. Usually, an exon encoding an important region of the protein (or an exon 5′ to that region) is interrupted by a positive selectable marker, e.g., neo, preventing the production of normal mRNA from the target gene and resulting in inactivation of the gene. A gene may also be inactivated by creating a deletion in part of a gene, or by deleting the entire gene. By using a construct with two regions of homology to the target gene that are far apart in the genome, the sequences intervening the two regions can be deleted (Mombaerts et al., 1991, Proc. Nat. Acad. Sci. U.S.A. 88:3084-3087).

Antisense and ribozyme molecules that inhibit expression of the target gene can also be used in accordance with the invention to reduce the level of target gene activity. For example, antisense RNA molecules that inhibit the expression of major histocompatibility gene complexes (HLA) have been shown to be most versatile with respect to immune responses. Still further, triple helix molecules can be utilized in reducing the level of target gene activity. These techniques are described in detail by L. G. Davis et al. (eds), 1994, Basic Methods in Molecular Biology, 2nd ed., Appleton & Lange, Norwalk, Conn., which is incorporated herein by reference.

Once the cells of the invention have been genetically engineered, they may be directly implanted into the patient to allow for the amelioration of the symptoms of disease by, for example, producing an anti-inflammatory gene product such as, for example, peptides or polypeptides corresponding to the idiotype of neutralizing antibodies for GM-CSF, TNF, IL-1, IL-2, or other inflammatory cytokines. Alternatively, the genetically engineered cells may be used to produce new tissue in vitro, which is then implanted in the subject, as described supra.

The use of the compositions and methods of the invention in gene therapy has a number of advantages. Firstly, since the culture comprises eukaryotic cells, the gene product will likely be properly expressed and processed to form an active product. Secondly, gene therapy techniques are generally useful where the number of transfected cells can be substantially increased to be of clinical value, relevance, and utility. Thus, for example, the three-dimensional culture described supra allows for mitotic expansion of the number of transfected cells and amplification of the gene product to levels that may be efficacious in treating congenital or acquired disease. Transplant of HLA matched cells, used banked cells, etc. are all advantages.

### (6) Production of Biological Molecules

In a further embodiment, the cells of the invention can be cultured in vitro to produce biological products in high yield. For example, such cells, which either naturally produce a particular biological product of interest (e.g., a growth factor, regulatory factor, or peptide hormone etc.), or have been genetically engineered to produce a biological product, could be clonally expanded using, for example, the three-dimensional culture system described above. If the cells excrete the biological product into the nutrient medium, the product can be readily isolated from the spent or conditioned medium using standard separation techniques, e.g., such as differential protein precipitation, ion-exchange chromatography, gel filtration chromatography, electrophoresis, and HPLC, to name but a few. A “bioreactor” may be used to take advantage of the flow method for feeding, for example, a three-dimensional culture in vitro. Essentially, as fresh media is passed through the three-dimensional culture, the biological product is washed out of the culture and may then be isolated from the outflow, as above.

Alternatively, a biological product of interest may remain within the cell and, thus, its collection may require that the cells be lysed. The biological product may then be purified using any one or more of the above-listed techniques.

One important application of the stem cells of the invention is the creation of feeder cell culture materials. The stem cells of the application can be used in the form of the feeder cell that remains alive, can produce growth factor and other materials for maintaining culture materials but do not divided or grow. The feeder cells are prevented from beginning or conducting a mitotic process by using irradiation, chemical treatment or other technique that can prevent such process. After performing such processes the feeder cells are alive and can function but will not divide or grow. In using feeder cells to culture the stem cells of the invention, the feeder cells provide growth factors to the growing totipotent, pluripotent or multipotent stem cells; however, growth factors can be added to the culture if the feeder cells are incapable of providing sufficient quantities. The feeder cells are grown and selected such that they express selected growth factors, for example, factors useful in the manufacture of neural, epithelial or other such desirable cell types and characteristics.

Preferably, the feeder cells are treated to prevent mitotic transformations or are inactivated prior to use. Preferably, feeder cells are inactivated using radiation or chemical treatment. Radiation useful for such transformation can include X-radiation, gamma radiation or electron radiation from appropriate sources. X-radiation can be used from electronic generation or from agents such as cobalt or cesium. Chemical treatments can be made with agents such as Mitomycin C. The resulting inactivated feeder cells can be cultured in culturing PGC's, preferably for 24 hours prior to culturing with a stem cell material. Feeder cell layers can be useful for both the isolation of stem cell lines from embryos and other sources and for the routine maintenance of established cell lines. Prior art used feeder layers prepared from mitotically inactivated fibroblasts. These are plated to give a uniform monolayer of cells onto which the stem cells are seeded. Feeder cell layers would be prepared from the Wharton's jelly obtained stem cells of the invention. Species-specific feeder cells can provide adequate growth conditions for successful culture development. As we have shown above cells can be rendered mitotically inactive by two means, exposure to irradiation such as gamma rays or by treatment with the drug mitomycin C. Fresh isolates can be taken on a regular basis to ensure that the cells are continually available. The stem cells can be isolated for feeder cell purposes, and other purposes, by obtaining the Wharton's jelly through dissection of the umbilical. Once isolated from the umbilical cord, the UCMS Jelly can be dispersed and suspended in an aqueous medium such as trypsin EDTA solution. Adding DMEM solution plus serum can neutralize the trypsin. The contents of the dish are transferred to a 10 ml conical tube. The tube is then centrifuged or permitted to settle large particulate materials. The stem cells in the supernatant can be plated with standard growth medium and maintained with conventional culture technique.

The use of the stem cells of this invention as a feeder cell in stem cell cultures provides a number of advantages. First, the cells are stem cells and provide growth factors that are applicable to other human stem cells from other sources such as embryonic sources, adult sources such as blood sources, adipose or fat sources and other human sources. Further, the use of human stem cells derived from UCMS Jelly provides a final cell culture in which the feeder cells do not prevent the use of the cultured stem cells from application in human use. In the prior art, many stem cell cultures were maintained on murine or other feeder cell lines preventing such cultures for use in humans due to the presence of non-human feeder cells. Such feeder cell cultures can be made using techniques disclosed in the following references, Van de Griend et al., “Rapid Expansion of Human Cytotoxic T Cell Clones: Growth Promotion by a Heat-Labile Serum Component and by Various Types of Feeder Cells”, (1984), Journal of Immunology Methods, 66:285-298; Robertson, E. J. (1987) in Teratocarcinomas and Embryonic Stem Cells: A Practical Approach, E. J. Robertson, ed. (Oxford: IRL Press), p. 71-112); and Fraley et al., Proc. Natl. Acad. Sci. USA, 80:4803, 1983.

The above specification, examples and data provide a complete description of the manufacture and use of the composition of the invention. Since many embodiments of the invention can be made without departing from the spirit and scope of the invention, the invention resides in the claims hereinafter appended.

### EXAMPLES

### Example 1

Purpose:

To isolate UCMS Jelly for use as a matrix.

To test the hypothesis that Wharton's Jelly will hold the useful stem cells in an undifferentiated state.

Method:

Umbilical cords were collected and the serosa opened. Two types of sample were prepared. One was adherent to the serosa, the other adherent to the vessels.

Each sample was exposed to mercaptoethanol, PBS, 10 mM MEDTA, 1 mM PMSI, 0.5% 2-mercaptoethanol and digested overnight. The sample was then dialyzed against water for 72 hours. The retentate had 40-80 microg/ml protein. Harvest procedure was adapted from Guerardel et al. (Biochem J 352: 449-463. 2000).

### Example 2

Purpose:

To identify an easily attainable source of potentially multi-potent stem cells that can be maintained in culture.

Method:

Induction of neural cells from UCMS cells: We utilized a procedure based on the method described by Woodbury et al. [2000] to induce UCMS cells to become neural cells. The UCMS cells were pre-induced by overnight treatment with basic fibroblast growth factor (10 ng/ml) DMEM and 20% fetal bovine serum. Neuronal differentiation was induced with 2% DMSO and 200 μM butylated hydroxyanisole in DMEM +2% fetal bovine serum. After 5 h, the media was modified for long-term induction by adding 25 mM KCl, 2 mM valproic acid, 10 μM forskolin, 1 μM hydrocortisone and 5 μg/ml insulin. By replacing this media every 36 hours we have maintained long-term cultures of the induced cells for longer than 1 month.

Immunocytochemistry was done by immunoperoxidase staining using standard methods. Briefly, cultured cells were grown on sterile glass cover slips in 24 well plates. Prior to immunodetection, they were washed briefly with PBS and the cells fixed by treating with methanol at −10° C. Slides were blocked with 10% normal blocking serum (derived from same species as the secondary antibody) in PBS for 20 min, washed with PBS, incubated with primary antibody in 1.5% normal blocking serum in PBS for 60 min (0.1 to 2.0 μg/mL depending on antibody). The slide was washed three times with PBS and incubated with an HRP-conjugated secondary for 15 min. Preparation of UCMS Whole-cell Lysates were made from UCMS cells by standard techniques using a lysis buffer (RIPA) consisting of PBS with 1% Nonidet P40, 0.5% sodium deoxycholate, 0.1% SDS and a protease inhibitor cocktail (1:500) (Sigma P8340). Lysis buffer was added to the culture dish with UCMS cells after washing with cold PBS 3 times. The culture dishes were then scraped and the lysate was aspirated into a syringe with a 21-gauge needle to shear DNA. The lysates were rocked in the cold for 1 h and centrifuged for 10 min at 10,000×g to remove insoluble material. Protein concentration was determined by the Micro BCA assay (Pierce). Typically protein concentrations of 1 μg/μL were obtained by this protocol. Immunoblotting: Solubilized proteins (10 μg per lane) were separated by SDS-PAGE under reducing conditions and transferred to nitrocellulose membranes by electrophoretic transfer in a tank system with plate electrodes. The membranes were blocked for 1 h at room temperature with 5% nonfat milk in Tris-buffered saline (TBS: 100 mM Tris, 0.9% NaCl, pH 7.5) containing 0.1% Tween 20. Membranes were incubated with primary antibody for 1 h at room temperature followed by 3 washes with 0.1% Tween/TBS. Membranes were incubated for 1 h at room temperature with the appropriate horseradish peroxidase conjugated secondary antibody diluted in 0.1% Tween/TBS. After four additional washes, with 0.1% Tween/TBS, the blots were visualized by chemiluminescene and recorded on radiographic film. 2D-electrophoresis: Protein (40 μg) from total cell lysates was precipitated by ice cold acetone and resuspended in 25 μL of sample buffer containing 62.5 mM Tris HCl pH 6.8, 2.3% SDS, 5% b mercaptoethanol, 10% glycerol and 0.01% bromophenol blue. Samples were loaded into capillary tube gels with an ampholyte range from pH 3 to 10 and were electrophoresed at 500 V for 10 min and 750 V for 3.5 h in a Mini Protean 2D Cell (BioRad). The second dimension separation was done using standard SDS-PAGE with an 8 to 16% gradient gel.

Results:

UCMS cells from neonatal calves were expanded as primary cultures. Initially they resembled flattened UCMS cells but with time round cells were observed growing on top of the UCMS cells. The round cells adhered to one another to form compact colonies. Within one hour multiple “neurites” were seen extending from many cells and the cell bodies became rounded and refractile. By four to five hours, some cells resembled bipolar or multipolar neurons and extended long processes that contracted similar processes from other neuron-like cells to form primitive networks. Growth cone-like swellings were seen at the ends of some of the processes. Cultured UCMS cells synthesized the catecholaminergic neuron marker, tyrosine hydroxylase.

After treatment with bFGF overnight and serum free media plus butylated hydroxyanisole and dimethylsulfoxide they assumed the morphology of neural stem cells e.g. a rounded cell body with multiple neurite-like extensions. Eventually some cells resembled bipolar or multi-polar neurons, and processes contacted each other to form networks. Expression of neuronal and glial cell specific proteins was produced in untreated UCMS cells. Both Western blotting and immunocytochemistry were used to determine the bFGF-treated neural stem-like cells and the more differentiated compact colonies.

Neuron specific enolase was detected in UCMS cells, the neural stem-like bFGF treated cells and in the more differentiated compact colonies at equal levels. TUJ1, an early neuron specific protein, was expressed in both the treated and bFGF-treated UCMS cells but not in the more differentiated colonies. Expression of TUJ1 was increased in the neural stem-like cells compared to the untreated UCMS cells. Likewise, glial fibrillary acidic protein (GFAP), an astroglial cell specific protein, expression was increased by treatment of UCMS cells with bFGF. Induced UCMS cells stained for neuron-specific enolase (NSE).

Conclusion:

Following the described procedure UCMS cells easily differentiated into neurons. The differentiated UCMS cells were characterized using immunocytochemistry and Western blotting. Untreated UCMS cells, in many cases exhibited positive staining for neural proteins. The study has produced cultures of Umbilical Cord (UCMS) cells that include cKit positive cells and myofibroblasts that express smooth muscle actin. The UCMS cells have telomerase activity and can be maintained in culture for extensive periods.

The UCMS cells are capable of differentiating along a neural program spontaneously. Induction speeds up this process and increases the number of UCMS cells that follow the neural program. After induction UCMS cells develop a neuron-like morphology with neurite-like processes and networks between cells. UCMS cells express protein markers for neural stem cells, mature neurons, astrocytes and oligodendrocytes.

Expressed neuronal markers included neurofilament (NF-M, 14 kD) and tau, a protein expressed in mature neurons.

The results show that UCMS cells from neonatal tissue may be a novel source of neural stem cells.

### Example 3

We have successfully propagated bovine and porcine BMS and UCMS cells. UCMS cells have been maintained beyond 55 passages and show no signs of decreased vigor.

The cells are derived from Wharton's Jelly matrix rather than cord blood because umbilical vessels are stripped from the cord before explant preparation and the cells are negative for markers of the hematopoietic lineage such as CD34 and CD45. This monoclonal antibody was specific for bovine cells and tested against the bovine UCMS cells by Western blot and immunochemistry.

### Example 4

The UCMS cells have been subjected to harsh environmental conditions such as prolonged exposure to room temperature, prolonged periods without media replacement and culturing in serum-free media. In the latter case they all become spherical and thrive and divide as suspension cultures.

### Example 5

In the Central Nervous System (CNS), two stem cell populations have been identified: ependymal cells and subventricular zone astrocytes. In culture, neural stem cells form clonal cell aggregates called “neurospheres” and embryonic stem cells form spherical embryoid bodies. UCMS cells have also been shown to form spherical aggregates in culture that resemble neurospheres.

When UCMS cells initially grow outward from explants two populations of cells are present—spherical or flat, mesenchyme cells. When the cells become confluent, they form white, spherical colonies that remain attached to cells below. The colonies look like ‘neurospheres’. Cells can be seen migrating out of the colonies, and the colonies grow in size over time. Occasionally they expand into a tube-like structure.

The cells within the colonies are very tightly adhered to one another. They can be mechanically dissociated with difficulty after prolonged trypsinization. When they are subsequently re-plated, the rounded cells grow rapidly to form new confluent monolayers and new colonies. The colonies have been sectioned and stained with hematoxylin and eosin. The colonies are noted to be heterogeneous with polyhedral cells, fusiform cells and small dark cells present. Elongated eosinophilic structures reminiscent of bone spicules are present.

### Example 6

We have injected of bovine BMS stem cells into the liver of fetal pigs (UCMS exp in progress). After 30-40 days in utero some of the fetal pigs were sacrificed. The bovine cells incorporated into many different tissues. Xenografted UCMS cells had taken up residence, divided and differentiated into a variety of cell types. This indicates that stem cells can incorporate themselves into virtually any tissue and differentiate appropriately.

Other pigs that were injected in utero with bovine BMS cells are currently alive and healthy.

### Example 7

Purpose:

To determine whether porcine UCMS cells could incorporate into rat nervous tissue.

Background:

UCMS cells were isolated from Wharton's jelly of neonatal bovine and porcine umbilical cord (UCMS). These cells can be propagated in culture without signs of decreased vigor. UCMS cell cultures contain cells that express smooth muscle actin indicating that, like bone marrow stromal cells, they are of the myofibroblast lineage. In addition, some cells are cKit positive suggesting that they can be activated by stem cell (pigUCMS) factor. When treated by the method of Woodbury et al. (2000) for inducing bone marrow stromal cells to become neural cells, UCMS cells undergo profound changes in morphology and many resemble bipolar or multipolar neurons. Neural proteins including neuron specific enolase, nestin, a neural-specific intermediate filament, TUJ1, a class III neuron-specific β-tubulin, GAP43, an axon-specific protein, CNPase, a marker for oligodencrocyte differentiation and glial fibrillary acidic protein (GFAP), an astroglial cell specific protein were detected based on immunocytochemical and Western blot analyses.

Method:

To determine whether porcine UCMS cells could incorporate into rat nervous tissue, UCMS cells loaded with fluorescent dye were injected in rat brains. After 2-6 weeks, the rats were sacrificed and their brains sectioned for analysis.

Results:

Most dye-loaded cells were found along the injection tract, however, a subset of the cells migrated away from the injection site and into the brain parenchyma. Immunocytochemistry was used to determine whether the UCMS cells differentiated into neurons or glia, or remained relatively undifferentiated.

### CONCLUSION

Cultures of UCMS cells include cKit positive cells. UCMS cells have telomerase activity and can be maintained in culture for extensive periods. UCMS cells are capable of differentiating along a neural program spontaneously. Induction speeds up this process and increases the number of UCMS cells that follow the neural program. UCM cells develop a neuron-like morphology after induction, with neurite-like processes and networks between cells. UCMS cells express protein markers for neural stem cells, mature neurons, astrocytes and oligodendrocytes. PUC cells can incorporate into nervous tissue and do not initiate a host-immune response when injected into rat brain cortex.

Together, these results suggest that UCMS cells can incorporate into nervous tissue and that they produce few or no cell surface antigens capable of initiating a host-immune response. Therefore UCMS cells may be a source of easily attainable, multi-potent stem cells that are more primitive than adult UCMS cells and thus may also have universal donor-like potential.

The specification, experimental section and description of the UCMS cell isolation, growth, transformation and use provide a basis for understanding the invention. The invention however should not be limited to the disclosure set forth above since a variety of embodiments can be obtained without departing from the spirit and scope of the invention. The invention resides in the claims hereinafter appended.

We claim:

1. A method for obtaining stem cells from an umbilical cord matrix comprising:
(a) fractionating the umbilical cord matrix source of cells, the source substantially free of cord blood, into a fraction enriched with stem cells, and a fraction depleted of stem cells, and
(b) exposing the fraction enriched with stem cells to conditions suitable for cell proliferation.

2. The method of claim 1 wherein the source of cell comprises umbilical cord Wharton's jelly.

3. A cultured isolate comprising stem cells isolated from an umbilical cord matrix source of stem cells, other than cord blood, the isolate comprising totipotent immortal stem cells.

4. A method of differentiating stem cells to a transplantable cell, the method comprising:
(a) obtaining a totipotent stem cell obtained from a umbilical cord matrix source of cells, the source other than cord blood; and
(b) exposing the stem cell to a differentiating factor to produce a transplantable cell.

5. The method of claim 4 wherein the transplantable cell is a hematopoietic cell.

6. The method of claim 4 wherein the transplantable cell is a mesenchymal cell.

7. The method of claim 4 wherein the transplantable cell is a neuro-ectodermal cell.

8. A method of treating a mammalian subject for alleviation of a disease symptom, the method comprising obtaining a transformed cell comprising stem cells isolated from a source of such cells derived from umbilical cord other than cord blood and transplanting that cell into a human subject requiring treatment provided by the transformed cell.

9. A method of introducing a foreign gene into a stem cell, the method comprising obtaining a totipotent immortal stem cell of claim 1 and contacting that stem cell with a transforming factor comprising a foreign gene.

10. The method of claim 9 wherein the transforming factor comprises a viral vector having a gene sequence foreign to the vector and native to the stem cell.

11. A method of generating a bank of mammalian stem cells from an umbilical cord matrix, the method comprising:
(a) fractionating the umbilical cord matrix into a fraction enriched with stem cells and a fraction depleted of cells; and
(b) culturing the fraction enriched with stem cells in a culture medium containing one or more growth factors, wherein the stem cells undergo mitotic expansion.

12. The method of claim 10 further comprising tissue typing, banking and expanding the totipotent umbilical cord mesenchyme cells needed.

13. The method of claim 10 further comprising differentiating the totipotent umbilical cord matrix cells in vitro.

14. The method of claim 10 further comprising genetically manipulating the totipotent umbilical cord matrix cells in vitro.

15. The method of claim 10 further comprising passaging the totipotent umbilical cord mesenchyme cells for at least 10 times and the umbilical cells remaining stable.

16. The method of claim 10 wherein the mammalian cells are from anay placental animal.

17. The method of claim 10 wherein the mammalian cells are human.

18. The method of claim 10 wherein the mammalian cells are porcine or bovine.

19. The method of claim 10 wherein the mammalian cells are equine or canine.

20. The method of claim 10 wherein the mammalian cells are rodent.

21. A method of transplanting the transplantable cell of claim 4, the method comprising:
culturing the totipotent umbilical cord matrix stem cells in a culture medium containing one or more growth factors wherein the stem cells undergo mitotic expansion.

22. The method of claim 21 further comprising:
culturing the umbilical cord stem cells in a culture medium containing one or more growth factors for inducing the production of stem and neural cells.

23. The method of claim 21 further comprising:
culturing the umbilical cord stem cells in a culture medium containing one or more growth factors for inducing the neural cells to undergo mitotic expansion.

24. The method of claim 21 further comprising:
culturing the neural cells in a culture medium containing one or more growth factors for inducing dopamine production in the neural cells.

25. The method of claim 21 wherein the neural transplantable cell is introduced into the substantia nigra region of the midbrain in a patient with Parkinson's disease.

26. The method of claim 21 wherein the neural transplantable cells are capable of producing dopamine.

27. A method of transplanting the transplantable cell of claim 21, the method comprising culturing the umbilical cord matrix stem cells in a culture medium containing one or more growth factors wherein the stem cells undergo mitotic expansion.

28. The method of claim 21 further comprising culturing the umbilical cord matrix stem cells in a culture medium containing one or more growth factors for inducing the production of fibroblast cells wherein the fibroblast cells undergo mitotic expansion.

29. The method of claim 28 further comprising introducing the fibroblast cells into a patient.

30. The method of claim 28 wherein the fibroblast cells have a homing ability for injured tissues and assist in tissue repair.

31. A purified preparation of human UCMS cells comprising:
(a) totipotential UCMS cells derived from Wharton's jelly; capable of proliferation in an in vitro culture for over one year;
(b) maintaining a karyotype in which all the chromosomes characteristic of the human are present and not noticeably altered through prolonged culture; and
(c) maintaining the potential to differentiate into derivatives of endoderm, mesoderm or ectoderm tissues throughout the culture.

32. The stem cells of claim 31 wherein the stem cells are capable of being typed, banked or expanded.

33. The method of claim 31 further comprising:
culturing the neural cells in a culture medium containing one or more growth factors for inducing neuron differentiation and maturation.

34. The method of claim 33 wherein the differentiated and mature neuron is introduced into the central nervous system of a patient.

35. The method of claim 33 further comprising:
culturing the neural cells in a culture medium containing one or more growth factors for inducing glial cell differentiation and maturation.

36. The method of claim 33 wherein the differentiated and mature glial cell is introduced into the central nervous system of a patient.

37. The method of claim 33 wherein the differentiated and mature glial cell is introduced into the spinal cord of a patient.

38. A stem cell culture comprising a stem cell population and a feeder cell population, the culture comprising:
(a) mammalian stem cells capable of proliferation in an in vitro culture for over one year;
(b) a feeder cell population comprising mammalian UCMS cells, said feeder cells incapable of beginning or conducting a mitotic process, but capable of providing growth factors;
(c) maintaining a karyotype in which all the chromosomes mammalian characteristics are present and not noticeably altered through prolonged culture; and
(d) maintaining the potential to differentiate into derivatives of endoderm, mesoderm and ectoderm tissues throughout the culture.

39. The stem cell culture of claim 38 wherein the stem cells are capable of being typed, banked or expanded.

40. The stem cell culture of claim 39 wherein the stem cells and the feeder cells are of human origin.

41. The stem cell culture of claim 39 wherein the matrix of UCMS is capable of delaying differentiation.

42. A method involving the use of the matrix or condition media to establish and maintain stem cells.

43. A method involving the use of the UCMS cells to generate transgenic or chimeric animals.
